(19) United States
(12) Patent Application Publication
(10) Pub. No.: US 2024/0115859 A1
(43) Pub. Date: Apr. 11, 2024

(54) METHOD AND SYSTEM FOR PROCESSING INPUT SIGNALS USING MACHINE LEARNING FOR NEURAL ACTIVATION

(71) Applicant: THE JOHNS HOPKINS UNIVERSITY, Baltimore, MD (US)

(72) Inventors: Cynthia STEINHARDT, Penn Valley, PA (US); Gene FRIDMAN, Baltimore, MD (US)

(73) Assignee: THE JOHNS HOPKINS UNIVERSITY, Baltimore, MD (US)

(21) Appl. No.: 18/277,179

(22) PCT Filed: Feb. 18, 2022
(86) PCT No.: PCT/US2022/017087

§ 371 Date: Aug. 14, 2023

Related U.S. Application Data
(60) Provisional application No. 63/150,829, filed on Feb. 18, 2021.

(51) Int. Cl. A61N 1/36; G06N 3/08; H04R 25/00

(52) U.S. Cl. CPC A61N 1/36038; G06N 3/08; H04R 25/606; A61N 1/0543

(57) ABSTRACT

A system and method for neural implant processing is disclosed. The method includes receiving, at a receiver of a neural implant, an input activation pattern; processing, by a front-end processing algorithm, the input activation pattern to produce a target population firing pattern for one or more neurons; and transforming, by a back-end processing algorithm, the target population firing pattern to a simulation pattern that induces a response with naturalistic timing. The neural implant includes a cochlear implant, a vestibular implant, a retinal vision prostheses, a deep brain stimulator, or a spinal cord stimulator.

### CROSS-REFERENCE TO RELATED APPLICATIONS

This application is the national stage entry of International Patent Application No. PCT/US2022/017087, filed on Feb. 18, 2022, and published as WO 2022/178316 A1 on Aug. 25, 2022, which claims the benefit of U.S. Provisional Patent Application Ser. No. 63/150,829, filed Feb. 18, 2021, both of which are hereby incorporated by reference herein in their entireties.

### GOVERNMENT RIGHTS

This invention was made with government support under grant NS092726 awarded by the National institutes of Health and grant DGE1746891 awarded by the National Science Foundation. The Government has certain rights in the invention.

### FIELD

This disclosure relates generally to method and systems for processing input signals using machine learning for neural activation.

### BACKGROUND

Cochlear implants (CIs) are arguably the most successful neural implant with nearly 40 years of innovation and over 300,000 users worldwide. CIs have been shown to significantly improve speech recognition and comprehension in children and adult users. However, they have had considerably less success in being able to restore pitch of sound. This poses major issue for CI users who speak tonal languages, such as Mandarin, who have difficulties with speech comprehension, and a lesser but significant quality of life deficiency for music appreciation.

In the cochlea, fine-timing of cochlear neuron firing is associated with pitch perception. In other applications, fine-timing depends on the particular neural system being considered. In cochlear afferent neurons, neurons will file action potentials at specific times as a stimulus is presented. Over numerous stimulations of sound, there will be some variability in which exactly the neurons fire. This variability is the degree of precision or fine-timing that is desired in cochlea implants. Pitch perception is important for tonal languages like Chinese and music appreciation. This has been a problem cochlear implants have attempted to address for over 20 years. In other neural implants, such as vestibular implants, lack of natural fine-timing has been associated with inaccurate restoration of eye movement.

Until recently, the focus of improving cochlear implants has been on preventing current spread from distorting perceived sound. Hardware innovations were implemented to minimize electrode distance from the modiolar wall to more directly target spiral ganglion neurons, and algorithms were modified to avoid electrical interference by ensuring no electrodes delivered current simultaneously; these improvements led to significant gains in fidelity of targeting neurons for spatial encoding of sound to give the percept in sound of a certain frequency. These improvements paired with the continuous interleaved sampling (CIS) strategy, modulation of amplitude of fixed-rate pulsatile stimulation to the envelope of sound, have led to highly accurate English speech comprehension in CI users.

Studies indicate that the inability to correctly convey pitch are the result of unrealistic CI-evoked timing of neural responses. Thirty-years of detailed studies produced a phenomenological model that reflects the full process of sound processing, including mechanical transduction and outer hair cell connectivity, and this model can produce fine timing for a cochlear neuron at any specified location along the basilar membrane.

However, this algorithm cannot process sound in real time even on a powerful desktop processor. It requires considerably longer to process the sound than the duration of the actual sound. Some studies have been performed on normal hearing subjects listening to computer generated tones using algorithms that deliver pulses with timing reflecting fine structure of sounds. These studies indicate that including timing information leads to better perception of tonal language (Mandarin).

While replicating exact timing of natural spiking has not been attempted per se, high-rate pulsatile stimulation was popularized as an improvement to CIS because it leads to more desynchronized, naturalistic neural responses. This stimulation strategy led to improved speech perception in noisy environments. However, a recent study showed that reducing the number of these high rate pulses by half while considering natural firing principles improves speech perception. A strategy with reduced number of pulses but more accurate fine timing could provide better perception.

### SUMMARY

According to examples of the present disclosure, a method for cochlear implant processing is disclosed. The method comprises receiving, at a receiver of a cochlear implant, an input natural sound pattern; processing, by a front-end processing algorithm, the input natural sound pattern to produce a target population firing pattern for a cochlea; and transforming, by a back-end processing algorithm, the target population firing pattern to a simulation pattern that induces a response with naturalistic timing.

Various additional features can be included with the method for cochlear implant processing including one or more of the following features. The front-end processing algorithm comprises a trained neural network. The trained neural network is a trained recurrent neural network. The trained recurrent neural network is trained to learn a sound wave-to-spiking relationship of a phenomenological model of the cochlea. The phenomenological model of the cochlea accounts for outer hair cell and inner hair cell contributions to firing, filtering effects, and non-linearities related to synaptic and axonal activation. The trained recurrent neural network is trained on a synthetic waveform data set and a speech command dataset. The trained recurrent neural network transforms sound pressure level (SPL) into spiking and firing rate over time for an auditory nerve fiber with low, medium, or high spontaneous firing.

According to examples of the present disclosure, a system for cochlear implant processing is disclosed. The system comprises a cochlear implant comprising a receiver that receives an input natural sound pattern; a front-end processing algorithm that processes the input natural sound pattern to produce a target population firing pattern for a cochlea; and a back-end processing algorithm that transforms the target population firing pattern to a simulation pattern that induces a response with naturalistic timing.

Various additional features can be included with the system for cochlear implant processing including one or more of the following features. The front-end processing algorithm comprises a trained neural network. The trained neural network is a trained recurrent neural network. The trained recurrent neural network is trained to learn a sound wave-to-spiking relationship of a phenomenological model of the cochlea. The phenomenological model of the cochlea accounts for outer hair cell and inner hair cell contributions to firing, filtering effects, and non-linearities related to synaptic and axonal activation. The trained recurrent neural network is trained on a synthetic waveform data set and a speech command dataset. The trained recurrent neural network transforms sound pressure level (SPL) into spiking and firing rate over time for an auditory nerve fiber with low, medium, or high spontaneous firing.

According to examples of the present disclosure, a method for neural implant processing is disclosed. The method comprises receiving, at a receiver of a neural implant, an input activation pattern; processing, by a front-end processing algorithm, the input activation pattern to produce a target population firing pattern for one or more neurons; and transforming, by a back-end processing algorithm, the target population firing pattern to a simulation pattern that induces a response with naturalistic timing.

Various additional features can be included with the method for neural implant processing including one or more of the following features. The neural implant comprises a cochlear implant, a vestibular implant, a retinal vision prostheses, a deep brain stimulator, or a spinal cord stimulator. The front-end processing algorithm comprises a trained neural network. The trained neural network is a trained recurrent neural network. The trained neural network is trained using clinical data, a phenomenological model, or both. The trained neural network comprises one or more convolution layers for retinal prosthesis analysis.

According to examples of the present disclosure, a system for neural implant processing is disclosed. The system comprises a neural implant comprising a receiver that receives an input activation pattern; a front-end processing algorithm that processes the input activation pattern to produce a target population firing pattern for one or more neurons and a back-end processing algorithm that transforms the target population firing pattern to a simulation pattern that induces a response with naturalistic timing.

Various additional features can be included with the system for neural implant processing including one or more of the following features. The neural implant comprises a cochlear implant, a vestibular implant, a retinal vision prostheses, a deep brain stimulator, or a spinal cord stimulator. The front-end processing algorithm comprises a trained neural network. The trained neural network is a trained recurrent neural network. The trained neural network is trained using clinical data, a phenomenological model, or both. The trained neural network comprises one or more convolution layers for retinal prosthesis analysis.

### BRIEF DESCRIPTION OF THE FIGURES

Various features of the embodiments can be more fully appreciated, as the same become better understood with reference to the following detailed description of the embodiments when considered in connection with the accompanying figures, in which:

FIG. 1 shows a system diagram of a front-end sound processing algorithm for a cochlear implant to process natural sound and produce a target population firing pattern for the cochlea according to examples of the present teachings. This pattern could then be transformed into a stimulation pattern that induces a response with naturalistic fine timing. A neural network will be used to learn the relationship between sound and firing rate from a realistic phenomenological model of the cochlea. In this disclosure, the performance was tested on a simplified problem, producing single auditory fiber responses to sine wave and step stimuli, because the CIS algorithm does not replicate firing for these inputs but the cochlear model does.

FIG. 2A, FIG. 2B, FIG. 2C shows a network architecture, where waveforms are transformed in spectrograms in MATLAB and each bin of the 400 Hz power band is run through the neural network to generate induced firing rate over time is shown in FIG. 2A, target outputs are generated by the Zilany model with smaller time bins is shown in FIG. 2B, and a recurrent neural network is used to turn each time bin into thirty-two firing rate predictions over time is shown in FIG. 2C, according to examples of the present teachings.

FIG. 3A and FIG. 3B show loss/performance with epochs, where the rms by the last epoch of training on networks with 25 to 1000 GRU nodes is shown on the left of FIG. 3A and the test performance of each of the trained models on 100 novel sine and step modulated waveforms is shown on the right of FIG. 3a and the best performance of each size network on the training and test data with the number of training epochs at which it best performed written above is shown in FIG. 3B, according to the present teachings. Error bars are SEM.

FIG. 4A and FIG. 4B show relative performance on sine and step waveforms, where input power signal (402, 404) and prediction (406, 408) and target firing response generate by Zilany model is shown in FIG. 4A and test performance across models on predicting responses to sinusoidal versus step inputs with networks trained for number of epochs of best overall performance is shown in FIG. 4B, according to examples of the present teachings. Statistics are two-sample t-test. *, p<0.1.

FIG. 5A, FIG. 5B, FIG. 5C, and FIG. 5D show performance on natural sounds, where best training performance on synthetic sounds of each model and test performance on natural sounds for same number of training epochs. *, p<0.1; ***, p<0.01 with paired t-test and stars above are true in all cases unless there is a shown exception is shown in FIG. 5A, test performance across models on predicting responses to natural inputs is shown in FIG. 5B, example natural inputs with word, GRU size and number of training epochs written above are shown in FIG. 5C and FIG. 5D, according to examples of the present teachings. Target response and inferred response of RNN are shown.

FIG. 6 shows a plot of run-time Evaluation of RNN. The ratio of run-time for calculating the response to a 0.5 second synthetic sound was measures across 10 runs with the Zilany model and the trained RNN one 1 CPU from a 2.4 GHz 8-Core Intel Core i9 Processor on a 2019 15-inch MacBook Pro.

FIG. 7 shows a map of firing rate changes in response to pulse rate train presentations given spontaneous activity S and pulse amplitude I according to examples of the present disclosure.

FIG. 8A, FIG. 8B, FIG. 8C, FIG. 8D, FIG. 8E, FIG. 8F, FIG. 8G, and FIG. 8H show effects of pulse on a silent neuron according to examples of the present disclosure.

FIG. 9A, FIG. 9B, FIG. 9C, FIG. 9D, FIG. 9E, FIG. 9F, FIG. 9G, and FIG. 9H show interactions of pulses with spontaneous firing according to examples of the present disclosure.

FIG. 10A, FIG. 10B, FIG. 10C, FIG. 10D, FIG. 10E, and FIG. 10F shows prediction of experimental vestibular afferent responses according to examples of the present disclosure.

FIG. 11A, FIG. 11B, FIG. 11C, FIG. 11D, FIG. 11E, FIG. 11F, and FIG. 11G show measurement plots of the use rules to achieve desired spike rates according to examples of the present disclosure.

FIG. 12 is an example of a hardware configuration for computer device, which can be used to perform one or more of the processes described herein, according to examples of the present disclosure.

### DETAILED DESCRIPTION

Reference will now be made in detail to example implementations, illustrated in the accompanying drawings. Wherever possible, the same reference numbers will be used throughout the drawings to refer to the same or like parts. In the following description, reference is made to the accompanying drawings that form a part thereof, and in which is shown by way of illustration specific exemplary embodiments in which the invention may be practiced. These embodiments are described in sufficient detail to enable those skilled in the art to practice the invention and it is to be understood that other embodiments may be utilized and that changes may be made without departing from the scope of the invention. The following description is, therefore, merely exemplary.

Generally speaking, a neural implant and a method of using the neural implant are disclosed where a first processing component, for example a front-end processing system or algorithm, receives an input, for example a sound for a cochlear implant, and produces a target firing pattern and a second processing component, for example a back-end processing system or algorithm, uses the target firing pattern and maps that to a simulation pattern that produces a pulse rate to deliver the desired firing pattern. For example, the front-end system, algorithm, or method and the back-end system, algorithm, or method can be implemented in a number of neural implants for different use cases. In some examples, the front-end processing system or algorithm and the back-end processing system or algorithm can also be implemented into a single system or algorithm. There are already a variety of neural implant on the market or in late-stage trials, including cochlear implants, vestibular implants, retinal vision prostheses, deep brain stimulators, and spinal cord stimulators. These devices take an input signal and transmit it to target neural tissue to overcome a deficit using pulsatile electrical stimulation. During signal processing, the input signal is (1) converted to a target neural firing pattern in the local tissue and (2) the target neural firing pattern is transformed into a stimulation paradigm considering the number of implanted electrodes and their proximity to the tissue in that specific implant. Other input signals can include, but are not limited to, an image for retina stimulation, an electrical signal that can mitigate pain signals, or signals from other brain regions, such as the hippocampus. The outputs of the front end that are used as inputs to the back end can also include, but are not limited to, sound to firing rate over time for cochlear implants, spike timing of individual neurons, and local field potentials oscillations for epilepsy treatment. The electrical neuromodulation can include, but are not limited to, pulsatile stimulation, transcranial direct current stimulation, galvanic stimulation. The neuromodulation can also take the form of a magnetic stimulation.

As further disclosed herein, a neuroprosthetic is disclosed that can take an input (e.g. sound, image, motion) to neural population firing as used in current neural prostheses (cochlear implant, etc.), future neural prostheses, or an input from another neural system (e.g. neural firing from visual cortex and send to another neural population e.g. frontal cortex). The use of a neural network, as described herein, provides for a faster processing capability that increases its usefulness in current and further neural prostheses.

According to examples of the present disclosure, a front-end processing system and method is disclosed that can be applied to any neural implant design, such as, but not limited to, a cochlear implant, a deep brain stimulation implant, an ocular implant, or a vestibular implant. A validated computational model is used that predicts the response of any cochlear afferent to an arbitrary sound. This model takes too long to execute to be used in real-time devices (e.g. 0.5 s sound takes approximately 1.5 s to execute). As disclosed herein, a recurrent neural network (RNN) machine learning algorithm is trained to compute the same transformation in much faster than real-time (e.g. same 0.5 s sound takes only 5 ms to execute on the same processor—100×faster than real time). Faster than real time means that it can be used in a neural implant to process incoming information as it comes in rather than post-hoc. In some examples, the RNN has 32 outputs to establish the time resolution of the neural firing. In some examples of the cochlear implant example, the RNN provides the target firing rate as a function of time for a single “average neuron” located in the 400 Hz location of the cochlea. The “average” firing behavior is estimated by simulating the responses of 50 neurons located in that region and averaging them. In another example, firing behavior may be simulated for the three types of neurons in the cochlea (high, low, and medium spontaneous activity fibers).

As described further below for the cochlear implant, and in some examples, an existing phenomenological model (Zilany model) is used to train the neural network. However, other models and/or clinical data can be used to train the neural network. In the retinal implant example, a neural network can be trained using test data obtained from a retina explanted from an experimental animal where random light patterns are directed onto it and a record from the retinal ganglion cells obtained. Thus, in this example, the trained neural network can transform the light patterns to ganglion cell firings.

In the cochlea, fine-timing of cochlear neuron firing is associated with pitch perception. The level of precision of the fine-timing for the cochlea is on the order of about 5 to about 10 ms in the healthy fibers. In other neural implants, such as vestibular implants, lack of natural fine-timing has been associated with inaccurate restoration of eye movement. Other existing prostheses can benefit from stimulation paradigms that induce realistic fine-timing of spiking, as well. For other implants, such as brain implants (e.g. Neurolink), the same front-end can be used to transform brain signals from one neural population into brain signals in another neural population. In this case, the front-end is part of an implant, for example, to improve performance on cognitive tasks. As disclosed herein, only 100 training examples with inputs and outputs were required to get less than 4% error in predicting neural responses. If this applies across neural systems, datasets for training the machine learning algorithm could be collected in a clinic instead of with a phenomenological model to perform the same front-end transformation for different neural circuits. Hundreds of trials of responses to sound in individual patients being implanted can be performed to tune the algorithm specifically to the patient.

Because firing is stochastic, complex, and asynchronous, it is slow to compute. The phenomenological model used herein required about 1.5 seconds to compute the response of one cochlear neuron to about 0.5 seconds of sound. For multiple neurons, this model will then take about 1.5*N seconds. Meanwhile, the computation time with RNNs is not dependent on the number of neural responses being predicted. Instead, it will depend on the number of nodes the algorithm is trained to use when performing the prediction of cochlear neural responses. In this disclosure, a network with a core calculation of 500 nodes is shown that is well within real-time capabilities, and accurately predicting one neural response only required 25 nodes. If the computation time of the disclosed algorithm increased linearly with the number of neural responses predicted, this would allow computation of 20 distinct neural responses in real time. Currently, cochlear implants only use 20 electrodes now, so this is well within real-time needs for a cochlear implant. Similarly, other devices on market use less than 20 electrodes, making this technique suitable for those applications, as well. Additionally, because the computation time of a machine learning algorithm increases slower than linearly, it is possible to compute more than 20 neural responses in real-time.

In some instances, there are two steps to assessing improvements with this front-end design in a cochlear implant. The first is to scale up the neural network to predict 20 neural responses across the cochlea. The cochlea performs spatial encoding of sound, and electrodes are places throughout the cochlea in approximately equal spacing and therefore equal spacing in frequencies—125 Hz, 1170 Hz, 2217 Hz, . . . 20,000 Hz. Stimulating across these locations gives the sensation of sound perception presently. As disclosed herein, a model is disclosed that predicts responses across neurons tuned to these frequencies.

Despite being able to restore speech perception with 99% success rate, cochlear implants cannot successfully restore pitch perception or music appreciation. Studies suggest that if auditory neurons were activated with fine timing closer to that of natural responses pitch would be restored. Predicting the timing of cochlear responses requires detailed biophysical models of sound transmission, inner hair cell responses, and outer hair cell responses. Performing these calculations is computationally costly for real time cochlear implant stimulation. Instead, implants typically modulate pulse amplitude of fixed pulse rate stimulation with the band-limited envelopes of incoming sound. This method is known to produce unrealistic responses, even to simple step inputs. As disclosed herein, a machine learning algorithm is used to optimize the prediction of the desired firing patterns of the auditory afferents in response to sinusoidal and step modulation of pure tones. A trained network that comprises 25 GRU nodes can reproduce fine timing with 4.4 percent error on a test set of sines and steps. This trained network can also transfer learn and capture features of natural sounds that are not captured by standard CI algorithms. Additionally, for 0.5 second test inputs, the ML algorithm completed the sound to spike rate conversion in 300×less time than the phenomenological model. This calculation occurs at a real-time compatible rate of 1 ms for 1 second of spike timing prediction on an i9 microprocessor.

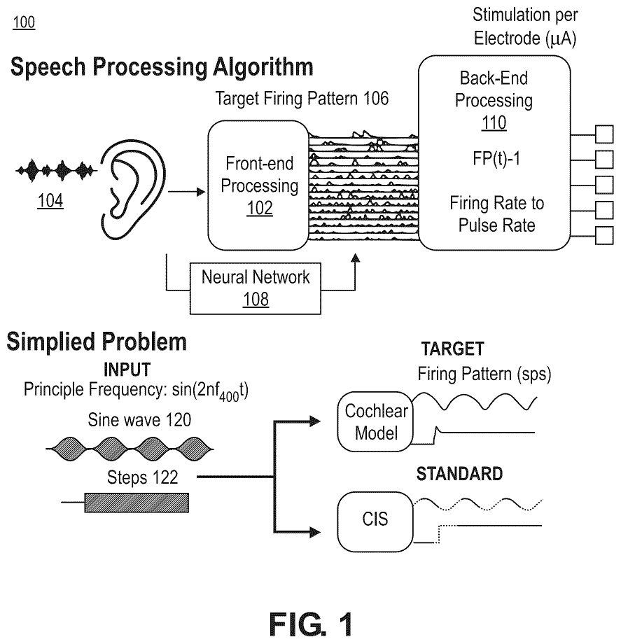

As disclosed herein, a front-end algorithm is used for cochlear implants that can transform any sound into the naturalistic fine timing of spikes for a fiber at real-time processing speeds using a machine learning (ML) approach, as shown in FIG. 1. As discussed herein, a machine learning algorithm, such as a neural network like a recurrent neural network (RNN), is trained to learn the sound-wave-to-spiking relationship captured in the validated Zilany 2014 version of the phenomenological model of the cochlea. The performance of the RNN in producing natural responses to sine waves and steps was evaluated. It was chosen to simplify the problem because the CIS algorithm fails to capture the complexity of the natural encoding of these stimuli. This front-end could then be included in a CI processing algorithm that (1) breaks sound into the power in spectral bands, (2) converts power to firing rate over time with a machine learning algorithm, and (3) converts induced firing rate into a pulse rate stimulation pattern, using equations relating pulse rates to induced firing rates.

FIG. 1 shows a system diagram 100 of a front-end sound processing algorithm 102 for a cochlear implant to process natural sound 104 and produce a target population firing pattern 106 for the cochlea according to examples of the present teachings. This target population firing pattern 106 is then transformed into a stimulation pattern that induces a response with naturalistic fine timing using a back-end processing algorithm 110. The front-end sound processing algorithm 102 can use a neural network 108 to learn the relationship between the natural sound 104 and firing rate based on the target population firing pattern 106 from a realistic phenomenological model of the cochlea. As disclosed herein, the performance is tested on a simplified problem, producing single auditory fiber responses to sine wave 120 and step stimuli 122, because the CIS algorithm does not replicate firing for these inputs but the cochlear model does.

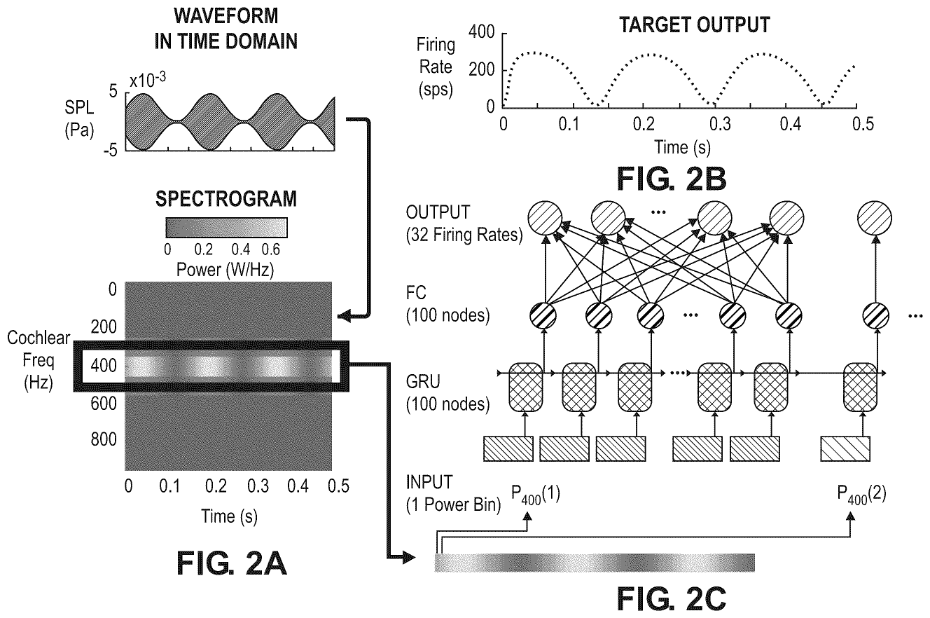

FIG. 2A, FIG. 2B, FIG. 2C shows a network architecture, where waveforms are transformed in spectrograms in MATLAB and each bin of the 400 Hz power band is run through the neural network to generate induced firing rate over time is shown in FIG. 2A, target outputs are generated by the Zilany model with smaller time bins is shown in FIG. 2B, and a recurrent neural network is used to turn each time bin into thirty-two firing rate predictions over time is shown in FIG. 2C, according to examples of the present teachings.

The generation of training and test waveforms is now discussed. The data used to train and test the model were synthetically generated sine waves and steps in power of a 400 Hz sinusoid, as shown in FIG. 1. All inputs were generated with signal of volume A_{mod} in dB, which was converted to sound pressure level (SPL) with Equation (1).

$${A}_{m o d} ( Pa ) = \sqrt{2} ( 2 {0}^{- 6} ( 10 \frac{{A}_{m o d} ( dB )}{20} ) ) \qquad ( 1 )$$

All inputs modulated a 400 Hz wave of the form:

s_{base}=sin(2πf_{princ}t),f_{princ}=400 HZ (2)

Sinusoidal modulation was performed with Equation 3

Sₛᵢₙₑ=Aₛₜᵢₘ(dm(1−A_{mod})sin(2πf_{mod} t+ϕ_{mod})+A_{mod})s_{base} (3)

where depth of modulation, dm=0.95, amplitude of modulation, A_{mod}, frequency of modulation f_{mod}, phase of modulation ϕ_{mod} and Aₛₜᵢₘ, stimulus amplitude were varied in the range in Table 1:

TABLE 1
Parameters for sinusoidal input generation

| | A_{mod} | f_{mod} (Hz) | ϕ_{mod} (radians) | Aₛₜᵢₘ (dB) |
|---|---|---|---|---|
| Min | 0.5 | 2 | 0 | 45 |
| Max | 0.9 | 40 | 2π | 95 |
| Steps | 8 | 10 | 5 | 10 |

Step modulation was performed with Equation 4, where A_{mod1} and A_{mod2}, the amplitude of each step and the shift, tmod were varied in the range of Table 2:

TABLE 2
Parameters for step input generation

| | A_{mod1} (dB) | A_{mod2} (dB) | t_{mod} (radians) |
|---|---|---|---|
| Min | 0.5 | 2 | 0 |
| Max | 0.9 | 40 | 2π |
| Steps | 8 | 10 | 5 |

Sₛₜₑₚ=(A_{mod1}+(A_{mod2}−A_{mod1})u(t−t_{mod}))s_{base} (4)

Additionally, performance of the model was tested on individual spoken word recordings from the training set of the Speech Command dataset. During testing, the waveforms were converted into power by using the spectrogram function in MATLAB with a hamming window of length 512, as shown in FIG. 2A. This produced 6.4 ms bins. The Zilany 2014 model was used to generate the natural firing rate over time in responses to these stimuli. The power was used as the input for the neural network, and the firing rate over time generated by the model was used as the target for training and evaluation, as shown in FIG. 2B and FIG. 2C. The Zilany model produced data every 0.2 ms. This difference in bin size was addressed when designing the neural network. For training, 100 synthetic waveforms were randomly sample from the data set, half sine waves and half steps. For testing performance on natural stimuli, 100 words from the Speech Command dataset were randomly selected.

A phenomenological model of the human auditory periphery (referred to here as the Zilany model) was developed over the last 30 years to replicate healthy auditory response to perception of any sound. This model accounts for outer hair cell and inner hair cell contributions to firing, filtering effects, and non-linearities related to synaptic and axonal activation. The model transforms sound pressure level (SPL) into spiking and firing rate over time for an auditory nerve fiber with low, medium, or high spontaneous firing, as shown in FIG. 2B. This disclosed model was used to create the training and test firing patterns using 50 ganglion cells in the physiologically observed ratio of low and high spontaneous activity fibers located at the 400 Hz position along the Basilar membrane. The neural responses (spikes/second over the duration of the sound stimulus) were used to create a dataset for testing and training the neural network.

Because this is an inherently “forwards-only” problem due to signal processing progressing from the eardrum to the ganglion cell firing pattern, a gated recurrent unit (GRU) can be used, which incorporates the memory of past network states to generate new inputs as the core of the network design for the task. This can account for effects of history, such as past spikes affecting proceeding spikes due to refractoriness. There were 32 firing rate values for every spectrogram time bin, so a fully connected layer was used to transform the outputs of the GRU layer into 32 outputs. This also allowed additional calculations to be made to adjust firing rate predictions within several milliseconds of one another that occur within one spectral bin. During this study, model size was assessed and used GRUs with 25, 50, 100, 200, 500, and 1000 nodes. A fully connected layer is used that reduced the GRU nodes to 32 outputs, as shown in FIG. 2C. The model was created using the Python Pytorch package. To train this model, the mean squared error (MSE) was used for backpropagation, using the “MSELoss” criteria.

To assess performance on the test and training data, the RMS was also used as a measure of error. During testing, the model was assessed on 100 waveforms (49 sinewaves). The model as also assessed for transfer learning to 100 speech command recordings. RMS was compared to the RMS between 10 predictions of the same response to sound with the Zilany model and compared to the RMS of the firing rate over time. The latter was transformed into a percent error.

Statistical testing between models and performance was computed with a paired t-test for comparing model size performance and a two-sided t-test when comparing performance on sinusoidal versus step modulation. Several performance metrics were used in addition to the prediction error. In addition to determining the minimum number of nodes necessary to perform the task of predicting responses to sinusoidal and step modulation, the computation time for the model was also assessed for the Zilany 2014 model and the neural network on simulation a 50-fiber response on the same computer in Python. Additionally, all size models were trained with between 500 and 250,000 epochs to determine the number of epochs that would be required to properly train each model and whether the loss was limited by network size. Run-time was evaluated on one CPU from a 2.4 GHz 8-Core Intel Core i9 Processor on a 2019 15-inch MacBook Pro when the trained RNN and Zilany model perform a prediction in response to the same 0.5 second sound 10 times. The ratio of speed was used as a metric in the results.

Based on previously published studies, cochlear implants successfully restore speech perception in non-tonal languages. However, implant users have difficulty with tonal languages and music appreciation, due to lack of accurate pitch perception. Studies indicate restoration of pitch perception requires fine timing of cochlear neuron firing. Standard implant algorithms, such as the CIS algorithm do not attempt to replicate this fine timing because it is unclear how to do this properly without a detailed computational model such as the Zilany model, which is computationally intensive to perform in real time.

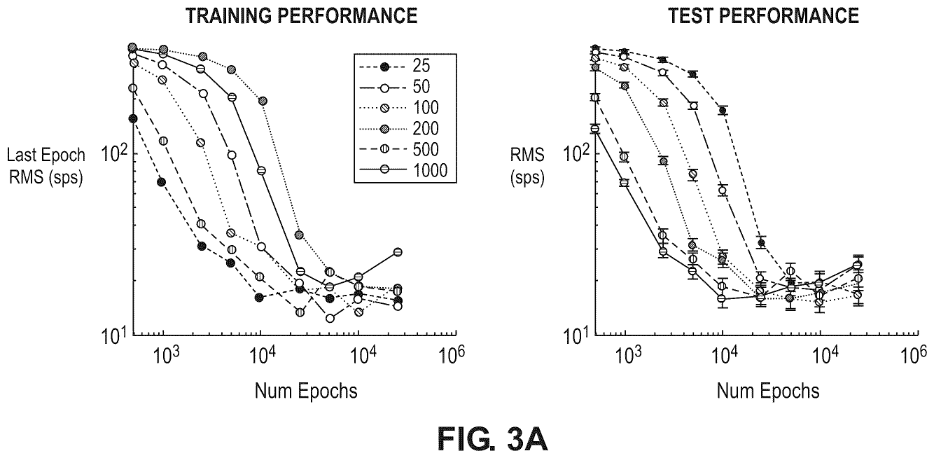

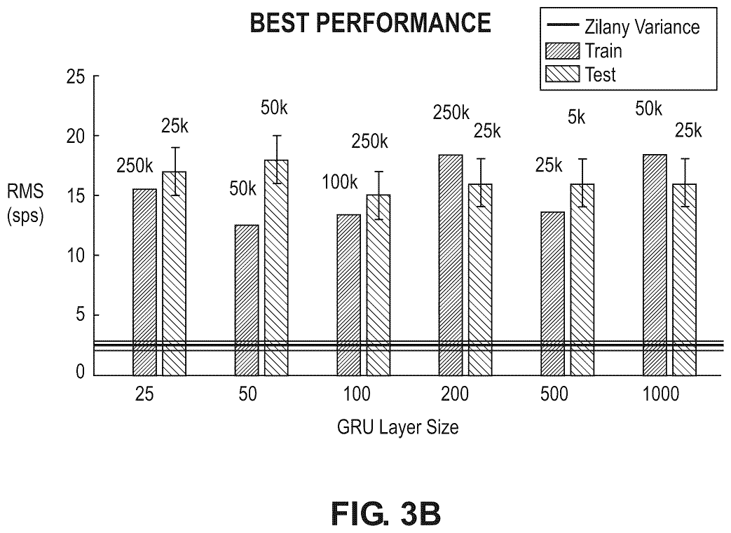

FIG. 3A and FIG. 3B show loss/performance with epochs, where the rms by the last epoch of training on networks with 25 to 1000 GRU nodes is shown on the left of FIG. 3A and the test performance of each of the trained models on 100 novel sine and step modulated waveforms is shown on the right of FIG. 3a and the best performance of each size network on the training and test data with the number of training epochs at which it best performed written above is shown in FIG. 3B, according to the present teachings. Error bars are SEM.

The performance of the model was tested on synthetic sounds. The training time and network size required to create an RNN that can perform this task was examined. The RNN contains a GRU layer and fully connected layer. The networks can be trained with as few as 25 GRU nodes and as many as 1000 GRU nodes for up to 250,000 epochs. The smaller models trained and reached the lowest training errors after fewer epochs (FIG. 3A left). However, all models converged to approximately the same performance by 250,000 training epochs. Larger models reached lower test error more quickly. However, by 250,000 training epochs, all model size performances were approximately the same (FIG. 3A right). It is speculated that there are fewer weights to adjust so these models converge more quickly to an optimization minimum. However, ultimately, even a 25-node GRU layer learned this transformation after a reasonable number of training epochs. Although the best performance occurred after different numbers of training epochs, depending on model size, all models had test performance comparable to training performance of approximately 10 sps error (FIG. 3B).

This was compared to the minimum achievable error, the rms between multiple simulations of the natural response to a sound with the Zilany model, which reaches a minimum of 3.0±0.2 (SEM) sps. Compared to the rms of the signal, the models on average have a test error of 4.20±0.03%.

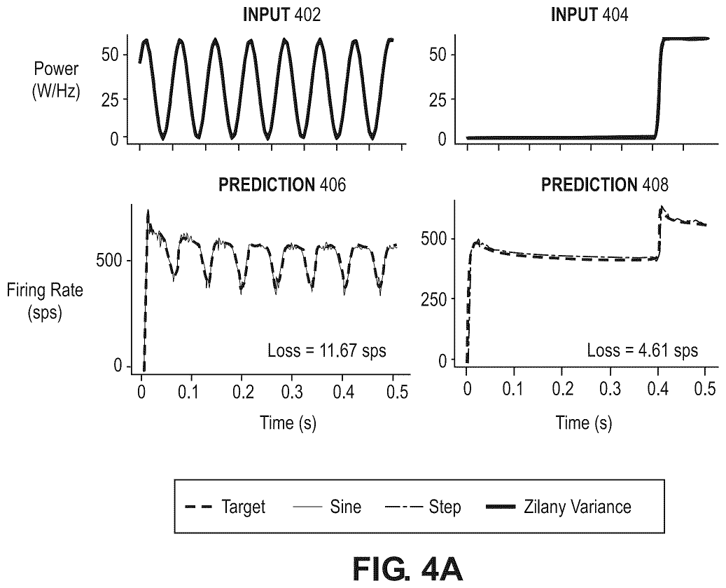

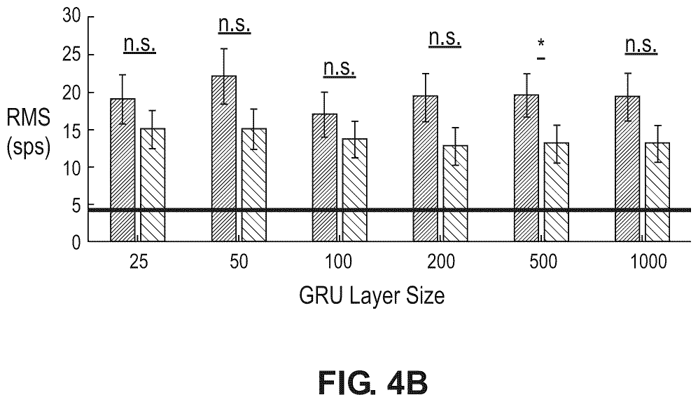

FIG. 4A and FIG. 4B show relative performance on sine and step waveforms, where input power signal (402, 404) and prediction (406, 408) and target firing response generate by Zilany model is shown in FIG. 4A and test performance across models on predicting responses to sinusoidal versus step inputs with networks trained for number of epochs of best overall performance is shown in FIG. 4B, according to examples of the present teachings. Statistics are two-sample t-test. *, p<0.1.

The models were trained to infer responses to both sinusoidal and step modulation (FIG. 4A). The RNN can generate both types of responses with high fidelity to the outputs generated by the Zilany model (FIG. 4A). The model appeared to predict step input responses more accurately. However, difference in performance were not statistically significant except for the 500 node GRU model (FIG. 4B). With more training, it is expected that performance will converge with a larger number of training epochs and more training data provided.

The performance of the model was also tested on natural sounds. The relative difficulty of learning responses was evaluated against natural sounds by testing the performance of the same model on natural response of the same 400 Hz auditory fiber bundle to audio recordings from the speech command. FIG. 4 shows relative performance on sine and step waveforms. (FIG. 4A) Input power signal and prediction and target firing response generate by Zilany model. (FIG. 4B) Test performance across models on predicting responses to sinusoidal versus step inputs with networks trained for number of epochs of best overall performance. Statistics are two-sample t-test. *, p<0.1.

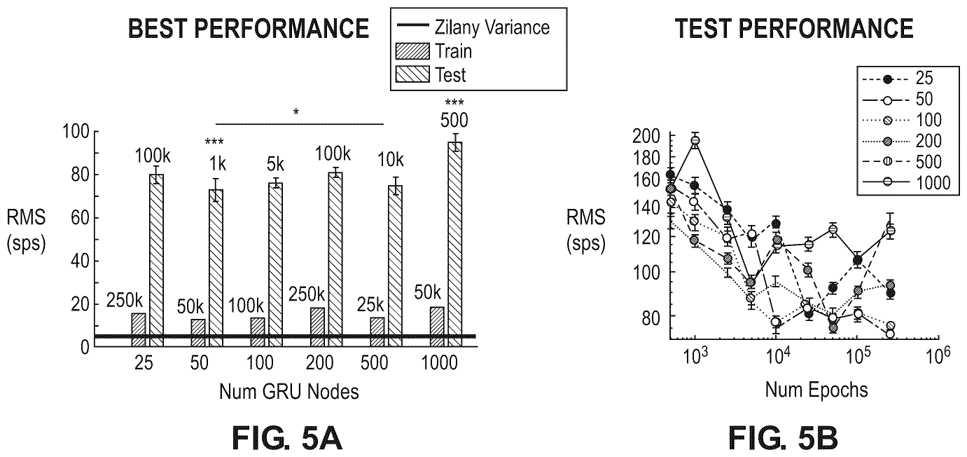

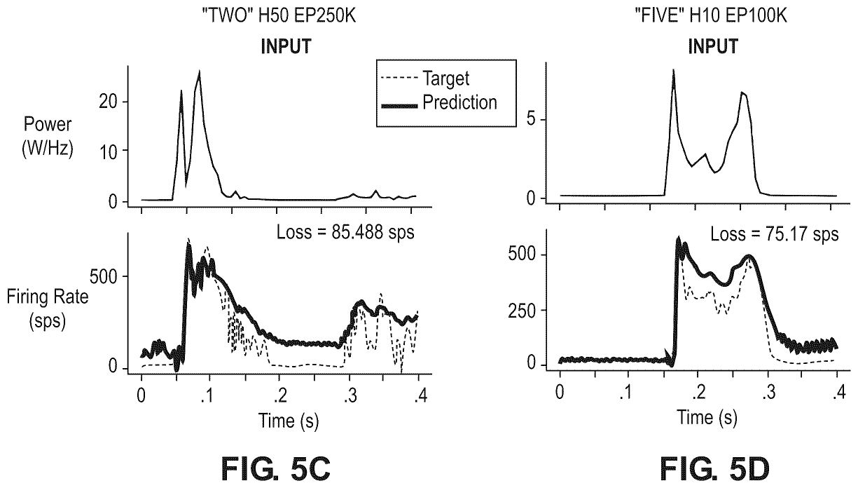

FIG. 5A, FIG. 5B, and FIG. 5C show the performance on natural sounds, where the best training performance on synthetic sounds of each model and test performance on natural sounds for same number of training epochs *, p<0.1; ***, p<0.01 with paired t-test and stars above are true in all cases unless there is a shown exception, is shown in FIG. 5A and the test performance across models on predicting responses to natural inputs is shown in FIG. 5B, and example natural inputs with word, GRU size and number of training epochs written above is shown in FIG. 5C. Target response and inferred response of RNN as shown.

It was found that rms error increases to about 80 sps across models, and smaller models outperform the largest, 1000-node RNNs significantly. The 50-node RNN outperforms all other models significantly but has similar performance to the 500-node RNN. The minimum percent error across models averaged 46.1±0.76%. It is speculated that the large model has not converged and learned the rules as accurately as the smaller models have with fewer weights and biases (FIG. 5A). This implies a larger network may not be necessary to learn this problem. However, longer training epochs are required to determine whether the larger networks could converge to a lower overall error level with more complex, natural sound responses. When measuring test performance across models with different numbers of training epochs, the best rms across models have not yet converged after 250,000 training epochs (FIG. 5B). This further indicates that more epochs are required to determine the relationship between best relative performance and model size on natural sounds. When observing differences between inferred response and target response the model captured non-linear transformations of the sound into firing rate (FIG. 5C left 0.05-1.5 s & 0.3-0.5 s, right 0.15-0.3 s). The model appeared to accumulate the most error for portions of response that were not scaled accurately. However, it captured complexities in shape that would not be captures with a CIS model, which converts the sound amplitude envelope to pulse amplitude. This result suggests that with more training epochs the RNN could capture both shape transformation and scaling accurately and learn natural image responses as well.

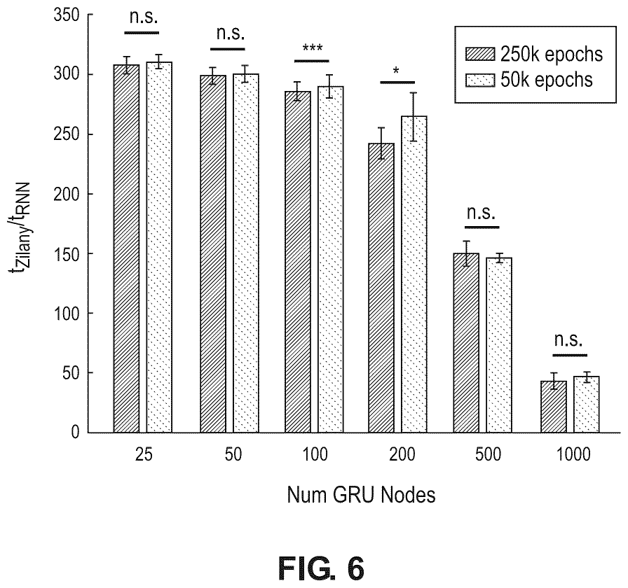

FIG. 6 shows a plot of run-time Evaluation of RNN. The ratio of run-time for calculating the response to a 0.5 second synthetic sound was measures across 10 runs with the Zilany model and the trained RNN one 1 CPU from a 2.4 GHz 8-Core Intel Core i9 Processor on a 2019 15-inch MacBook Pro.

The potential of these RNNs to be used in a real-time implementation were evaluated. The Zilany model required 1.47±0.01 seconds to predict the neural response of a single fiber to a 0.5 second sound. The 25-node network required 4.73±0.02 milliseconds. This improvement is plotted as a ratio of time to perform the task with the Zilany model over the time to perform the task with the RNN. The RNN was 335.4±4.54 times faster with a 25-node network trained with 50,000 epochs. The performance was evaluated with a minimum 50,000 epochs, because the performance of the RNNs converged by 50,000 epochs across models on the synthetic data. So, models of these size produced reasonable predictions of responses. The number of training epochs did not significantly influence run time for most models, as shown in FIG. 6. For models with a GRU layer with less than 200 nodes, run-time was approximately the same. As the model approached 100 nodes, the relative gain in computation speed was significantly reduced (FIG. 6). As performance was better when the RNN has less than 200 nodes (FIG. 5A), It is not anticipated requiring a network that is less than 200 times faster than the Zilany model. At these speeds, the model can perform a computation in approximately 1/100 of the length of the stimulus. If it is assumed that this processing speed scales with sound size, because the GRU steps is an iterative process, it is anticipated these computation speeds to be within the range of real-time.

The computation speed was evaluated on a 2019 MacBook Pro with an Intel Core i9 with 2.4 GHz Processor (1909980HK). These processors are clocked at 478 GFLOPS. The modern microcontrollers in an Apple iPhone XS perform at 487.5 GFLOPS, indicating this RNN should be able to perform at similar speed with modern cell phone microprocessor technology. These results show creating a real-time front-end for a cochlear implant that is used to generate realistic target responses. To use this disclosed front-end to the benefit of patients, algorithms also use accurate transformation of a predicted firing pattern to a stimulation pattern that can evoke this firing patter in actual neurons. These algorithms also incorporate complexities of how stimulation parameters, such as pulse amplitude and rate affect induced firing rate.

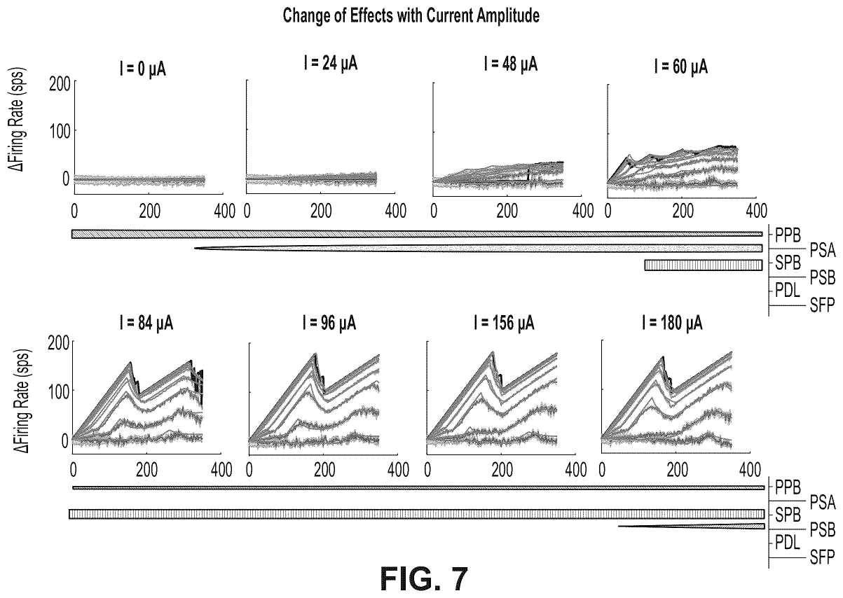

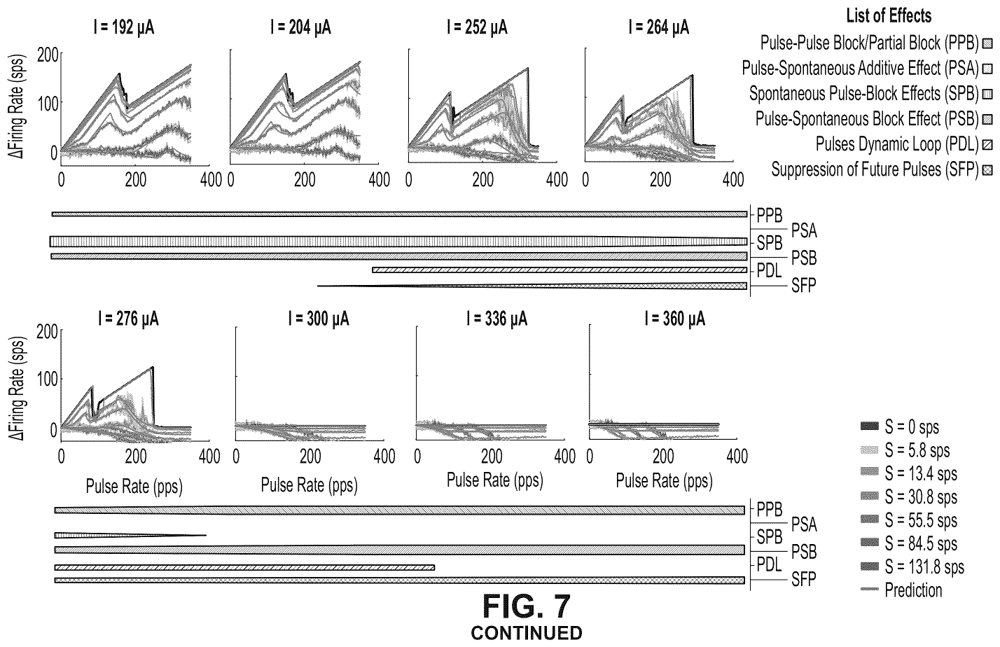

The back-end algorithm and/or system can be modeled based on one or more of the following considerations. FIG. 7 shows a map of firing rate changes in response to pulse rate train presentations given spontaneous activity S and pulse amplitude I according to examples of the present disclosure. The simulation was performed at each spontaneous rate and current amplitude. The change in firing rate (from the spontaneous rate) is shown with pulse rate to keep induced firing rates on the same scale. The prediction using the parameterized equations is shown for comparison. On the right the change in effect magnitude is shown by effect. Each row shows a transition from one effect to the next. The effects prevalent for a given range of amplitudes are shown in the left column. PPB: Pulse-pulse block/partial block, PSA: pulse-spontaneous addition, SPB: spontaneous-pulse block, PSB: pulse-spontaneous block, PDL: pulses dynamic loop, SFP: suppression of future pulses. Non-zero spontaneous rates were generated by selecting average EPSC rates from 0.25 ms (˜131 sps) to 8 ms (˜5 sps).

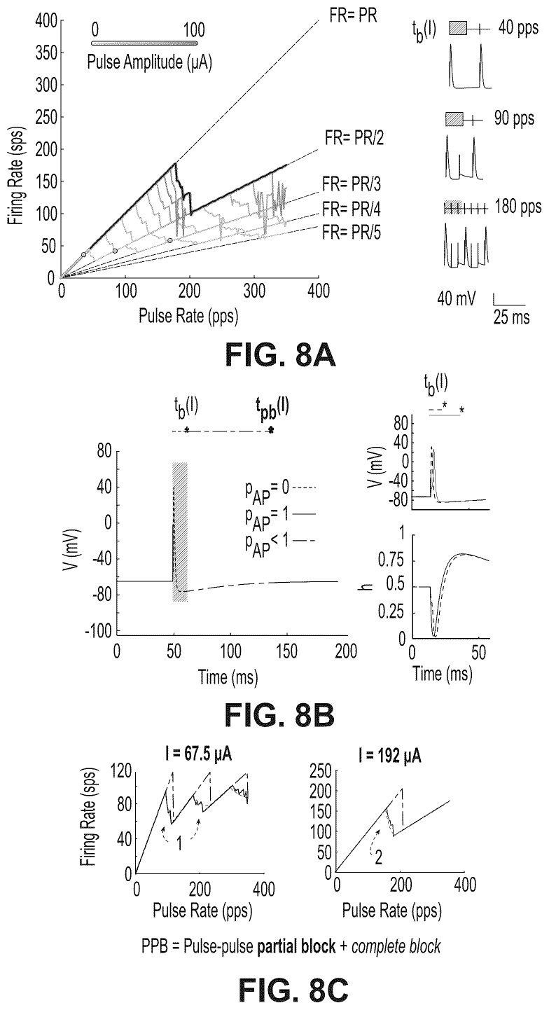

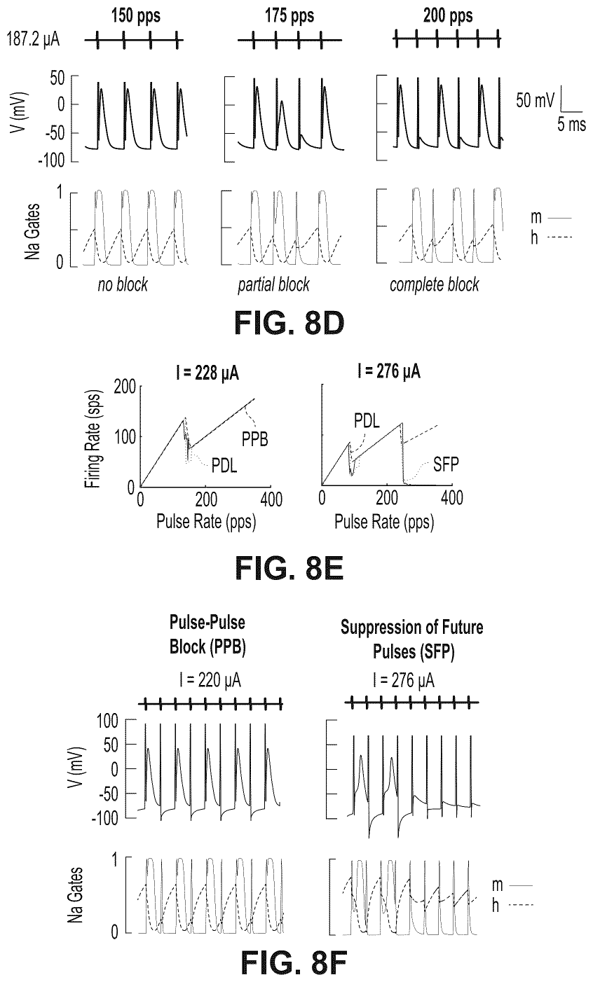

FIG. 8A, FIG. 8B, FIG. 8C, FIG. 8D, FIG. 8E, FIG. 8F, FIG. 8G, and FIG. 8H show effects of pulse on a silent neuron according to examples of the present disclosure. FIG. 8A shows the length of the block zone t_{b} changes with I. For one I, this block window leads to the firing rate reducing to subharmonics of the pulse rate depending on the ratio of the inter-pulse-interval and t_{b}. This effect is shown for I=60 μA (dots). FIG. 8B shows the after-pulse window pulses change axon channel states fully blocking pulses up to t_{b} ms after the pulse. In the recovery period, there is a Partial Elimination (PE) zone where a subsequent pulse would be blocked with a probability that decrease to zero if pulses are t_{pb} ms after the previous pulse. As I increases from threshold levels (dash) to higher I, sodium dynamics (h) reach states for firing more quickly, changing the length of the block and PE zone. FIG. 8C shows the block window and the block and partial block window described by equations compared to simulated relationship (see Methods below). 1. PE window decreases with subharmonic jump. 2. PE window increases with I. FIG. 8D shows the input pulses, voltage trace, and dynamics of the sodium channel m-gate and h-gate that drive the PPB and SFP effect. FIG. 8E shows at high I, the jump from FR=PR to FR=PR/2 creates a Pulse Dynamic Loop (PDL) that causes 1/3 or less of pulses to produce APs until the pulse rate increases, changing the dynamic paper so FR=PR/2 (left). A Suppression effect occurs at pulse rates that would at lower I produce FR=PR/2 leading to FR=0 (right). FIG. 8F shows the input pulses, voltage trace, and dynamics of the sodium channel m-gate and h-gate that drive the PPB and SFP effect.

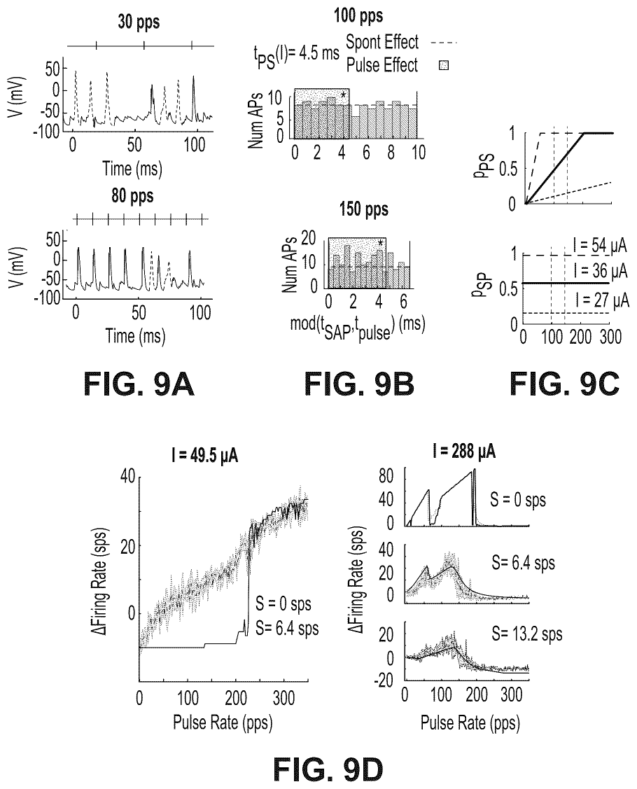

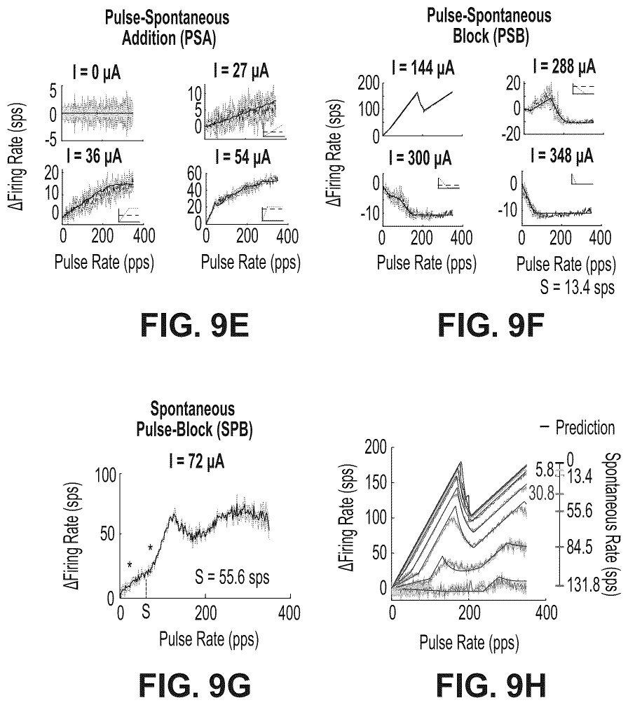

FIG. 9A, FIG. 9B, FIG. 9C, FIG. 9D, FIG. 9E, FIG. 9F, FIG. 9G, and FIG. 9H show interactions of pulses with spontaneous firing according to examples of the present disclosure. FIG. 9A shows interactions of spontaneous spikes with pulse-evoked activity at 30 pps and 80 pps. Spontaneous spikes are in green. FIG. 9B shows that at high and low amplitudes a pulse will interact with multiple spikes or a spike may interact with multiple pulses. Time of spontaneous activity compared to pulses takes an approximately uniform distribution between pulses, having equal chance of effecting all pulses (p_{SP}). As ρ decreases, the after-pulse window affects a greater portion of spontaneous activity (p_{PS}), so p_{PS}(ρ). As current increases both probability of pulses affecting spikes (P_{PS}) and spikes affecting pulses (P_{SP}) increase together. The probabilities fall between 0 to 1. FIG. 9C shows the presence of even a small amount of spontaneous activity (6.4 sps) leads to smoothing of pulse=induced effects leading to facilitation even at pulse rates below the rate required for typical pulse-pulse facilitation. FIG. 9D shows that this also leads to reduction and eventually disappearance of PDL effects as S increases to 13.2 sps. FIG. 9E shows facilitation effects can be explained with p_{SP} and p_{PS} increasing to 1. High amplitude blocking can be explained with p_{SP} and p_{PS} returning to 0. FIG. 9H shows the main spontaneous-pulse effect is blocking at mid-range I. The same pattern produced with S=0 is observed but with S lower firing rate and smoothed out partial elimination zones.

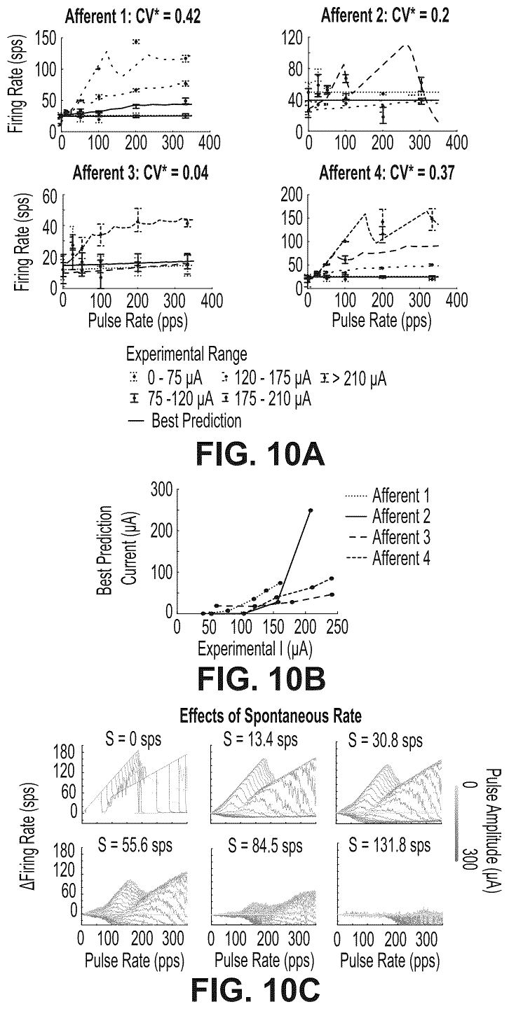

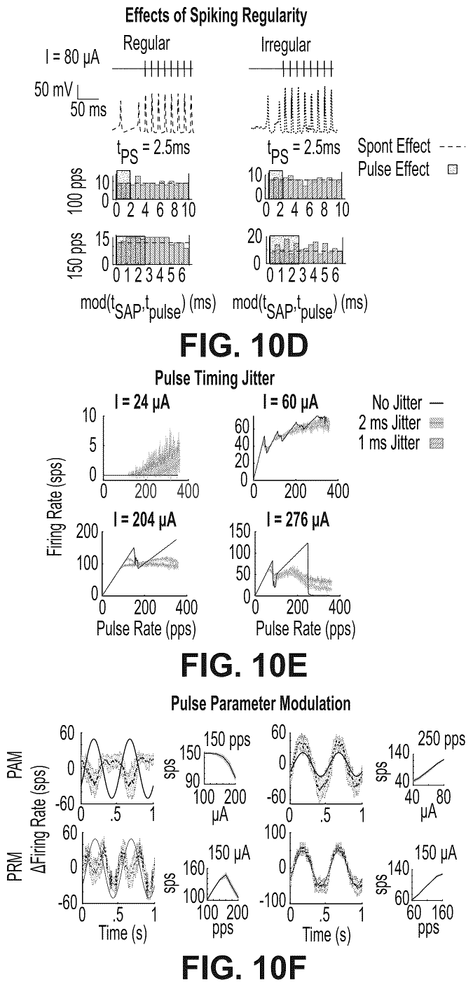

FIG. 10A, FIG. 10B, FIG. 10C, FIG. 10D, FIG. 10E, and FIG. 10F shows prediction of experimental vestibular afferent responses according to examples of the present disclosure. FIG. 10 A shows four afferents varying from regular (CV*=0.04) to irregular (CV*=0.42) in response to pulsatile stimulation with 0 to 240 μA. Predictions are shown in matching color to the data (dashed). Predictions used the same parameterization with respect to I and S. S was chosen as the firing rate immediately prior to the pulse block. Only the value of I was chosen to best fit the experimental data. FIG. 10B shows the relationship between I_{pred}, the I of best fit and the experimental value delivered at the electrode for each afferent. FIG. 10C shows that spontaneous activity has a massive effect on induced firing rate. The same currents lead to addition versus blocking depending on S. Each square shows the firing rate at the same amplitude with S=0 to S=131 sps. FIG. 10D shows regularity does not significantly affect the probability of pulse-spontaneous activity interactions for pr>S. So, the simplifying assumption can be made that regularity does not have a large influence. FIG. 10E shows that pulse delivery timing often occurs with a jitter. The effect of a jitter of 1 ms std and 2 ms std was tested compared to pulses with no jitter. It can be seen that the jitter smooths the partial elimination zone significantly, similarly to spontaneous activity. FIG. 10F shows Pulse rate modulation (PRM) and pulse amplitude modulation (PAM) with sinusoids was simulated on the model with modulation frequency of 1 Hz. The predicted induced pulse rate for sinusoidal pulse rate and pulse amplitude) modulation compared to predicted changes. For given current range and fixed pulse rate or vice versa the prediction of our equations for change in firing rate (bottom right).

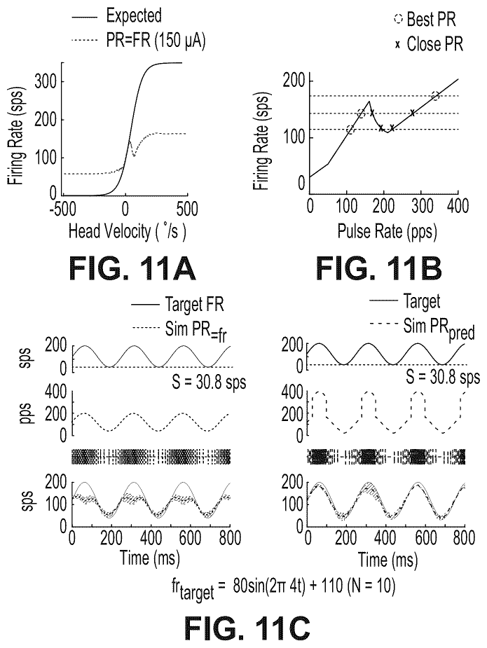

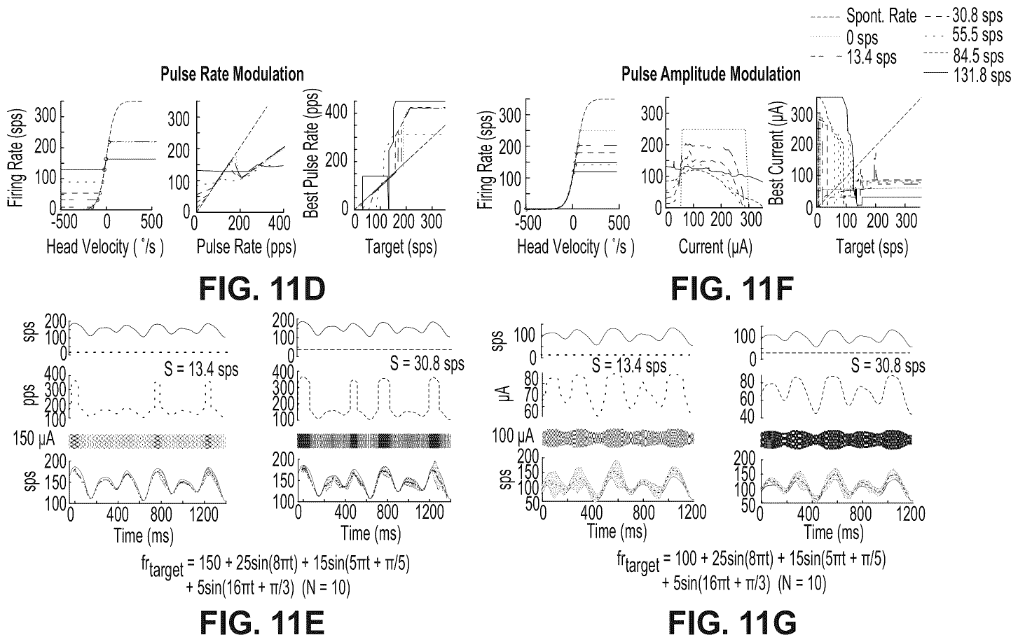

FIG. 11A, FIG. 11B, FIG. 11C, FIG. 11D, FIG. 11E, FIG. 11F, and FIG. 11G show measurement plots of the use rules to achieve desired spike rates according to examples of the present disclosure. With the assumption of one-pulse-to-one-spike mapping between pulse rate and spike rate, FIG. 11A shows that the resultant spike rate is nonlinear and highly limited in its ability to deliver the desired sensation of head velocity. FIG. 11B shows that using the rules described herein, a set of pulse parameters (xs) that produce a desired firing rate in a given neuron can be modeled and generated. A minimal power consumption algorithm was used that finds the minimum pulse rate and pulse amplitude for achieving a desired firing rate (circles). FIG. 11C shows a plot of target sinusoidal change in spike rate for 30 sps neuron. Below, the change in pulse parameters as a function and as pulse delivery and the resulting change in firing rate compared to the target based on simulations in the biophysical model. The results for the one-to-one mapping of pulse rates results in a nonlinear response of the neuron with a limited range of activity (left). The same target faithfully reproduced using the predicted nonlinear pulse presentation pattern. FIG. 11D shows a plot where the larger range of firing rates can be produced with PRM using the corrected strategy delivered to neurons with various spontaneous rates in same as previous plots. The achievable head velocities (left), the pulse rate to firing rate mapping (middle), and the optimal stimulation strategy compared to the expected one-to-one mapping (right). FIG. 11E shows analogous plots to FIG. 11C showing the same arbitrary stimulus reproduced by applying the optimal strategy for neurons with two different spontaneous rates (13 and 30 sps). FIG. 11F and FIG. 11G show analogous plots to FIG. 11D and FIG. 11E for PAM.

Experimental Stimulation Paradigms

Pulses were applied using an isolated pulse stimulator (A-M systems), which delivered biphasic pulses with 150 μs stimulation phases and no interphase gap. The same single unit stimulation paradigm used in physiology experiments was replicated in silico on the biophysical model. During each pulse block, 1 s pulse trains were delivered at rates from 25 to 300 pps. These rates were chosen because they fall within the physiological range of vestibular afferent firing. Fixed-rate pulse blocks were delivered at a fixed pulse amplitude in order from lowest to highest pulse rate. There were typically four repetitions of each pulse rate block before increasing the pulse rate. For several there were only three repetitions. For most pulse blocks there was a 500 ms or 1 s window between blocks. The maximum current was set to 80% of the minimum value that caused visible facial muscle activation in response to pulses delivered at 300 pps. Pulse rate experiments were performed with a fixed current amplitude and repeated at amplitudes from 25%, 50%, 75%, 87.5%, and 100% of maximum amplitude.

Data Analysis

The assay of neural responses is firing rate in response to blocks of pulses. Therefore, induced firing rate was measured as the number of APs that occurred from the onset of the first pulse in a pulse block to the offset of the last pulse in the block divided by the length of that time window. There were noticeable experimental differences in spontaneous activity before and after pulse blocks. Therefore, spontaneous activity was measured as the firing rate in the window preceding a pulse train, excluding the first 50 ms, if the window occurs after another pulse block. This was done to avoid remaining effects from the previous pulse train. Many stimulation paradigms assume a linear relation between pulse rate and firing rate. To test this hypothesis, data from all repetitions of pulse rate blocks at a single current amplitude were fit with the line best fit with a y-intercept equal to the average spontaneous rate (S): fr=m pr+S. The slopes of best fit are compared to the unity line, the slope if each pulses produced one AP.

Data Fitting with Equation fr=ϕ(pr,I,S)

Simulations revealed predictable, smooth transitions between effects of pulses with the change in pulse amplitude and spontaneous rate. These effects were captured through an equation fr=ϕ(pr,I,S) (Eq.5) explained further below that takes measurable and controllable values as inputs: pulse rate pr, pulse amplitude I, and spontaneous rate S. The equation captures effects from facilitation to blocking at 0 to 350 μA as fitted to a single irregular afferent simulation. Only the magnitude of parameters changes as a function of I and S to produce these effects.

The experimental data were fit by using the measured values of pr and S and optimizing on predicted current, I_{pred}. I_{pred} was chosen as the value that minimized the two-dimensional rms between the data at 25 to 300 pps and predictions. The two-dimensional error was taken between a prediction fr_{pred}=ϕ([1:300] pps,I_{pred},S) and the 8 experimental values. The (x,y) error for each of the 8 sampled pulse rates (k) was taken as the minimum distance between the closest point on fr_{pred} and the kth pulse rate-firing combination (ρₖ,frₖ):

$${rms}_{ρ fr} = {\sum }_{k = 1}^{8} {min}_{ρ} {\{ \sqrt{{( ρ - {ρ}_{k} )}^{2} + {( ϕ ( pr , {I}_{pred} , S ) - f {r}_{k} )}^{2}} \}}_{ρ = 1}^{300} \qquad ( 6 )$$

The total error for I_{pred} was the sum of the error at each of the 8 points.

The only restrictions on I_{pred} were the value that minimized the rms error described above and that the value was weighted by the size of I_{pred}:

$$ϵ = {rms}_{ρ fr} + . 5 \frac{{I}_{p r e d}}{200} \qquad ( 7 )$$

The second rule was enforced because many pr-fr combinations are the same at high and low I values. Also, between 100-200 μA, the relationship is approximately static. With the sparse sampling of pulse rate, this weighting helped assure I_{pred} was not overestimated. Note, all model errors reported on the figure are standard rms measured at the pulse rates tested in the experimental data.

Biophysical Modeling of Vestibular Afferents

Vestibular afferents were simulated using a biophysical model to study the effects of electrical stimulation on vestibular afferents Past work from the lab showed this model can replicate experimental firing rates and changes in firing rate with pulsatile and direct current stimulation.

An adapted version of the Hight and Kalluri model can be used for the modeling. In brief, Hight & Kalluri showed that vestibular firing can be simulated accurately by assuming cells have the same shape and size. Type I and Type II vestibular afferents are modeled as differing only in channel expression and EPSC magnitude (K). Spontaneous rate can be set by changing the average inter-EPSC arrival interval (m).

The membrane potential (V) varies as:

$$\frac{dV}{dt} = \frac{1}{( {C}_{m} S ) ( {I}_{Na} + {I}_{KL} + {I}_{KH} + {I}_{leak} + {I}_{epsc} + {I}_{stim} )} \qquad ( 8 )$$

where in addition to the current from each channel, membrane potential is influenced by the EPSCs arriving at axon (I_{epsc}) and the injected current (Iₛₜᵢₘ). The system of equations represents each cell as a single node with overall surface area, S=1.1·10⁻⁵ cm² and capacitance Cₘ=0.9 mF/cm². Each channel is voltage-gated and dependent on a conductance, an open state and a closed state variable: Na (g_{Na}, m, h), KH (g_{KH}, n, p), KL (g_{KL}, w, z). The electrode was stimulated at 2 mm from the simulated afferent which causes the firing threshold around 56 μA for a typical neuron.

The simulations used to parameterize the equations were performed on a single model of an irregular vestibular afferent. It was found that conductance values of g_{Na}=13 mS/cm², g_{KH}=2.8 mS/cm², and g_{KL}=1 mS/cm² and EPSCs with K=1 and m=1.3 ms matched previously published experimental findings at pulse rates from 25 to 300 pps. These conductance values were used for all irregular afferent simulations.

For studies with the effects of spontaneous rates on firing. The channel conductance values were kept the same but m was set to 0.25, 0.5, 1, 2, 4, and 8. To model the axon with no spontaneous activity, EPSCs was not added.

Additionally, the effect of firing regularity was assessed on induced firing rate. The irregular neuron (fr=36.6±0.9 sps, CV=0.57, where CV is Coefficient of Variance), was modeled with K=1, and m=1.65 ms. A conductance matched regular neuron (fr=33.8±0.4 sps, CV=0.09) was also modeled with g_{Na}=13 mS/cm², g_{KH}=2.8 mS/cm², and g_{KL}, =0 mS/cm², K=0.025, and m=0.09 ms.

The effects of conductance values on the pr-fr mapping with current amplitude was tested. Conductance values were used that produced firing rates similar to those observed in a previous in vitro experiment with and without exposure to DC current: 24.50 g_{Na}=7.8 mS/cm², g_{KH}=11.2 mS/cm², and g_{KL}=1.1 mS/cm², K=1. m was again varied from 0.25 to 8 ms.

No evidence of pulsatile stimulation affecting the hair cell was found, so all DC hair cell related effects (adaptation, the non-quantal effect, etc.) were not activate in these simulations. The simulation is run using the Euler method to update all variables through each of the channels.

Simulated Pulsatile Stimulation Experiments

The experiment conducted was repeated in silico with finer sampling of current amplitudes and pulse rates. Electrodes were simulated as being placed 2 mm from the vestibular afferent axon. In addition to the pulse rates used experimentally, pulse rates from 1 to 300 pps in steps of 1 pps were delivered for 1 second. Five repetitions were performed for each current amplitude, spontaneous rate, and pulse rate combination. Pulse amplitude was varied from 0 to 360 μA in steps of 12 μA and used to parameterize equations values. Interpolation between these values was used to create a smooth function for predicting induced firing rates.

This combination of experiments was repeated on the irregular neuron, regular neuron, and low conduction/in vitro neuron. It was also repeated for all values of μ to map how these effects change with different levels of spontaneous activity.

Jitter Experiment

To assess the effect of jittered pulse delivery time on induced firing rate, the same simulation was performed. Instead of delivering perfectly timed pulses, a gaussian noise term with standard deviation of 1 ms or 2 ms is added to the exact pulse timing to simulate delay or advancement in the delivery of regularly scheduled pulses (FIG. 10E).

Pulse Rate and Amplitude Modulation

To test how these rules apply to sinusoidal modulation, as used in various prosthetic algorithms, PRM and PAM was simulated within a common range for vestibular prostheses. Pulse rates were modulated by steps of 20 or 50 pps/μA around values where non-linearities or linearities were observed in the one-second fixed pulse rate experiments (FIG. 10F). Sinusoidal PRM and PAM modulation was simulated for the same afferent with a 42 sps baseline firing rate (FIG. 10F).

Predictive Equation

The observed effects at the axon were transformed into equations that depended on measurable or controllable variables: pulse amplitude (I) delivered from the electrode, pulse rate, as inter-pulse interval (ρ), and spontaneous rate (S). Equations were not optimized for real-time usage. The goal was to create equations that captured the observed effects and their mechanisms without dependence on time. The equations were made to reflect mechanism. Variables were shown to vary with I and S. They were best fit to each I-S combination of the simulated afferent. All variables that were best fit are marked with “′” in the following equations.

Pulse-Pulse Interactions

Pulse-Pulse Block/Partial Block (PPB)

The most substantial effect of pulses in the absence of EPSCs is pulses blocking following pulses from becoming APs. The visible refractory period after a pulse is driven by an underlying change in channel dynamics that leads to a zone where all pulses are blocked followed by a zone of exponentially decreasing blocking effects until pulses have zero probability of being blocked (FIG. 9A, FIG. 9B, and FIG. 9C). Because the following pulses are blocked, the pulses within the block zone do not extend these effects, leading to the induced firing rate being a subharmonic of the pulse rate:

$$f {r}_{PP} = \frac{( \frac{1}{ρ} )}{ceil ( \frac{{t}_{b} ( I ) ′}{ρ} ) + PE ( I , ρ )} \qquad ( 9 )$$

where the length of the full block zone t_{b}(I) changes with I (FIG. 7, FIG. 8B). t_{b} is large when I is small (due to channels driving the membrane towards resting state) and when I is large (due to the pulses causing large changes that push channels into unnatural states). At I around 100-200 μA in silico, t_{b} remains at approximately the same minimum values, as pulses are strong enough to consistently drive firing but not strong enough to cause extreme changes in channel states. The ratio of ρ to t_{b} determines what fraction of pulses are blocked, so the pulse rate at which 1/2 or 1/3 of pulses become APs is easily calculable as (n/t_{b})pps, where n is the denominator of the fraction. This effect is captured with division by the ceil( ) term.

After t_{b} until t_{pb}(I), the subsequent pulse is not blocked with certainty, but the probability of being blocked decreases with time. This zone is called the partial elimination (PE) zone. t_{pb} changes with I similarly to t_{b} (FIG. 7B and FIG. 7C). PE was implemented with respect to the transition point between each bend (n) at which the firing rate changes from pr/n to pr/(n+1) due to the observed changing in length of PE with each transition or bend in the pulse rate-firing rate relationship. Because transitions happen at each n/t_{b} pps pulse rate, the pulse rate at the start of PE for that transition can never be less than (n−1)/t_{b}. Therefore, the length of the PE zone as a fraction p′_{bp} times t_{b} is expressed as:

$${t}_{p b} ( n ) = \frac{{t}_{b}^{′}}{n - min \{ \text{.99} , {p}_{p b}^{′} )} \qquad ( 10 )$$

As ρ decreases such that n>1, p′_{pb} becomes an increasingly larger (FIG. 9C-1). This is due to a change between n and n−1 at large n being equivalent to a smaller time between pulses. The length of t_{pb} does decrease as n increases.

PE is implemented such that between

$$\frac{{t}_{b}}{n}$$

and t_{pb}(n), the term takes values that linearly decrease from 1 to 0 as the pulse rate approaches

$$\frac{n}{{t}_{p b}} .$$

This term adds to the ceil( ) term so that the transition is not abrupt from fr=pr/n to fr=pr/(n+1) but instead has an exponential decay from one harmonic line to the next (FIG. 9C). The strength of PE grows with I, which could be well characterized with the scaling κ:

$$κ = {( I - \frac{α′ ( S )}{300} )}^{4} \qquad ( 11 )$$

$$PE ( I , ρ ) = \{ {\sum }_{n = 1}^{N} min \{ \begin{matrix}1 , ( 1 + κ ( I , S ) ) ( 1 - \frac{ρ - \frac{{t}_{b}^{′}}{n}}{( {t}_{pb} ( n ) - \frac{{t}_{b}^{′}}{n}} ) \} & , & {t}_{pb} ( n ) < ρ < {t}_{b}^{′} / n \\ 0 & , & else\end{matrix} \qquad ( 11 )$$

At midrange I, the scaling effect with I causes lower prs to transition to the next subharmonic (fr=pr/(n+1)) but it never causes a harmonic to skip to over n+1. As I increases, such as at I=192 μA, this leads to a sharper transition from one line to the next (FIG. 9C). This effect is implemented with the min term such that PE£1. When spontaneous rate was increased, it is found that κ, the scaling up of PE effects, occurs at a lower I due to the spontaneous activity distorting and expanding the timing and likelihood of pulses causing large changes along the axon for smaller changes in membrane voltage. This effect is embedded in the equation through the α′ which increase with S.

At very high I (I>204 μA in the mapping) suppression effects occurred that caused axonal firing to shut down in longer, unnatural dynamic loops. Two main effects at high amplitudes (Pulse Dynamic Loop (PDL) and Suppression of Future Pulses (SFP)) were implemented as variations of the PE function.

Pulse Dynamic Loop (PDL)

At the transition from n=1 to n=2, with no spontaneous activity, it can be seen that the transition overshoots and comes returns to n=2 during the PE time (FIG. 9E, FIG. 9F). This was modeled with a similar function with reverse directionality of exponential decay:

$$for {t}_{p b} ( 1 ) < ρ < {t}_{b}^{′} , {PE}_{1} = ceil ( κ ( I , S ) ( \frac{ρ - {t}_{b}^{′}}{( {{t}_{p b} ( 1 )}^{′} - {t}_{b}^{′} )} ) ) \qquad ( 13 )$$

Suppression of Future Pulses (SFP)

Additionally, instead of the pulses within the immediate refractory period being blocked, the delivery of an additional pulse pushes axonal dynamics to a state of full suppression after n=2. Again, it can be seen that this transition is an exponential decay to fr=0 as opposed to a sharp drop (FIG. 9E, FIG. 9F). This rule is implemented as:

$$for ρ >= {t}_{p b} ( 2 ) , {PE}_{2} = ( 200 / {β}^{′} ( S ) ) {( 1 - \frac{ρ - \frac{{t}_{b}^{′}}{2}}{( {{t}_{p b} ( 2 )}^{′} - \frac{{t}_{b}^{′}}{2} )} )}^{3} \qquad ( 14 )$$

When spontaneous activity is included, it can be seen that, with larger spontaneous activity, the slope of this decay is exceedingly slower. This effect is enforced by β′(S), which increases from 1 to 250, when S=130 sps.

With these terms only, the induced firing rate simplifies to:

$$f {r}_{PP} = \frac{( \frac{1}{ρ} )}{min \{ 2 , ceil ( \frac{{{t}_{b} ( I )}^{′}}{ρ} ) \} + PE\_ 1 + PE\_ 2 )} \qquad ( 15 )$$

The min term assures smooth transition to fr=0 without the bends that would typically occur at pulse rates that are multiples of 1/t′_{b}.

Pulse-Spontaneous Interactions

Pulse-Spontaneous Additive (PSA), Pulse-Spontaneous Block (PSB), and Spontaneous-Pulse Block (SPB).

Regularly timed pulses break up time into windows of length ρ in which relative affects of pulses on spontaneous activity and vice versa can be approximated. This effect with mod(tₛ, φ is numerically implemented. This shows that spontaneous spikes are distributed uniformly between 0 and ρ ms after a pulse. So, pulses affect some fraction of the evenly distributed spontaneous activity, and all pulses are affected to some level by the ongoing spontaneous activity. As pr increases, the time between pulses is smaller but the probability of a spontaneous AP is equally likely, so a greater portion of spontaneous activity is affected per pulses. The probability of spontaneous activity affected by pulses is called, p_{PS}. The probability of pulses being affected by spontaneous activity and producing APs is p_{SP}. A simple approximation of these interactions would be:

$${p}_{PS} = min \{ 1 , \frac{ρ - {t}_{PS} ( I , S )}{ρ} \} \qquad ( 16 )$$

where t_{PS} is the time after a pulse when spontaneous APs are blocked.

$${p}_{SP} = min \{ 1 , \frac{( {t}_{S P} S )}{T} \} \qquad ( 17 )$$

where t_{SP} is the time after a spontaneous AP when pulses are blocked, and T is the total length of the time window. t_{SP} and therefore p_{SP} only depends on I, as spontaneous rate will remain the same for a neuron. With this approximation, t_{PS} will only depend on I and p_{PS} will increase linearly with the pulse rate until reaching 1. To simplify fitting, p′_{PS} and p′_{SP} are fitted directly depending on I. Spontaneous rate did not have large effects on these variables. Implementation would likely not be largely affected if these variables were kept the same for any spontaneous rate.

These pulse-spontaneous interactions took two forms. At low amplitudes, they worked together to produce facilitation of pulses at amplitudes that a silent axon could not produce activity (FIG. 9A, FIG. 9B, FIG. 9C, FIG. 9D, FIG. 9E, FIG. 9F, FIG. 9G, and FIG. 9H). By I=54 μA, both probabilities reach 1, representing the transition from pulses facilitating with spontaneous activity to blocking interactions (FIG. 9B and FIG. 9E). At very high I, the same equations taking the same shape can be used to describe pulses blocking spontaneous activity and spontaneous activity blocking pulses (FIG. 9F). These spontaneous activity effects are linked to EPSC facilitating or being facilitated by pulses into becoming APs, or pulses becoming large enough that underlying EPSC activity can quickly transition the axon to suppressed state. fit p′_{PS} and p′_{SP} both increases to 1 sigmoidally around 50 μA. The blocking effects of pulses on spontaneous activity start around 156 μA and are significantly dependent on spontaneous activity. The spontaneous activity scaling down pulse-induced firing changes rapidly around 290 μA without strong dependence on S.

Spontaneous-Pulse Full Block Effects

Between about 50 and 290 μA, spontaneous activity blocks pulses to a more limited degree. These effects are likely due to spontaneous APs and not EPSCs, as the quantities directly relate to S. The largest effect is that, as S increases, the same pr-fr relationships occur as in silent neurons but reduced by S (FIG. 10G and FIG. 10H). Voltage traces indicate this is due to spontaneous activity blocking pulses, and pulses are only shown to block spontaneous activity at significantly larger amplitudes. Thus, S is subtracted from fr_{P}. Additionally, there is a bend in the PFR (FIG. 10G). As described above, until ρ=t_{b}(I), the slope of the PFR should be 1. Prior to this bend, the slope is significantly lower, and afterwards it is 1. It is found that pulses are relatively weak, such that below approximately PR=S, pulses are often blocked by spontaneous APs, while, for PR>S pulses seem to alter dynamics so that multiple pulses survive between spontaneous APs (FIG. 10A). This non-linearity is fitted as an additionally slope until S that increases with I and is scaled down with S(δ).

Facilitation ends around 50 μA, but this threshold current varies with S, so it is called I_{fac}(S) and include facilitation as:

$$For I < {I}_{frac} ( S ) , {fr}_{p} = {p}_{PS}^{′} PR , PR < ( 0.8 / {p}_{SP}^{′} ) = {p}_{SP}^{′} {fr}_{PP} , PR > ( 0.8 / {p}_{PS}^{′} ) \qquad ( 18 )$$

$$For I > {I}_{frac} ( S ) , \begin{matrix}{fr}_{p} = {δ}^{′} ( I , S ) PR , & PR < S \\ = {p}_{SP}^{′} {fr}_{PP} - S - {fr}_{PP} ( S ) + {δ}^{′} ( I , S ) S , & PR > S\end{matrix}$$

$$\begin{matrix}{fr}_{S} = S - {p}_{PS}^{′} PR , & I > 156 μA \\ = S & else\end{matrix} \qquad ( 19 )$$

$$fr = max \{ 0 , {fr}_{S} ] + max \{ 0 , {fr}_{P} \}$$

The max term assures each term does not go negative if blocking effects exceed S or pr.

In summary, a real-time front-end for a CI could be created using an RNN. The RNN could be of a size of less than a 100-node GRU layer and a fully connected layer and perform the transformation of natural sound to sinusoidal and step neural response prediction with less than 5 percent error. Additionally, the relationships between sound and predicted firing pattern on this simplified task transfers to natural sound and captures a number of non-linearities in the transformation of sound into firing rate encoding by the cochlea. These RNNs can run over 300 times faster than the only phenomenological model that exists and can accurately produce natural cochlear responses to sound. These RNNs can run at real-time speeds on the microprocessors that exist in modern cell-phone technology. This approach produces neural implants that generate neural responses with the fine timing of natural population responses in the body and can be used to help in restoring pitch perception in CI users.

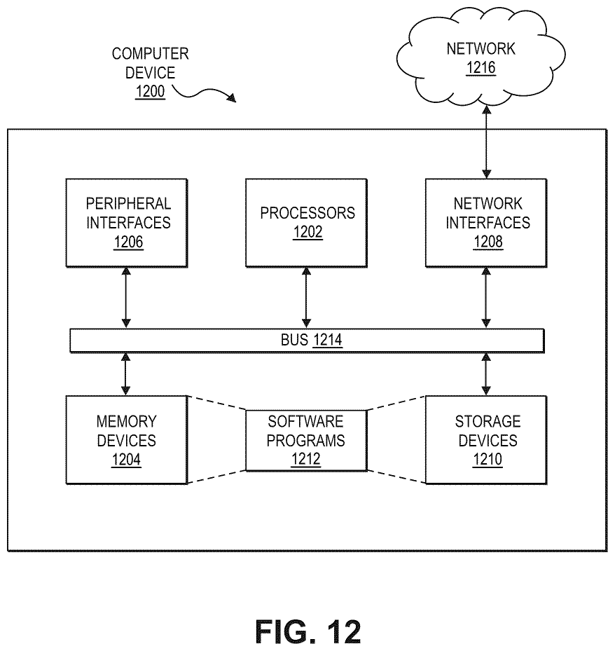

FIG. 12 is an example of a hardware configuration for computer device 1200, which can be used to perform one or more of the processes described above. Computer device 1200 can be any type of computer devices, such as desktops, laptops, servers, etc., mobile devices, such as smart telephones, tablet computers, cellular telephones, personal digital assistants, etc., or high power microcontrollers used to implement firmware such as ones used in hearing aids, cochlear implants, or other computer-powered devices. Some of these microprocessors include dedicated neural network hardware capability specifically designed to implement artificial neural networks and RNNs described here. These computer devices would allow easier and more efficient implementation of the front-end neural networks described here. As illustrated in FIG. 12, computer device 1200 can include one or more processors 1202 of varying core configurations and clock frequencies. Computer device 1200 can also include one or more memory devices 1204 that serve as a main memory during the operation of computer device 1200. For example, during operation, a copy of the software that supports the above-described operations can be stored in one or more memory devices 1204. Computer device 1200 can also include one or more peripheral interfaces 1206, such as keyboards, mice, touchpads, computer screens, touchscreens, etc., for enabling human interaction with and manipulation of computer device 1200. A data or communication bus 1214 provides data pathways between the various components of the computer device 1200.

The computer device 1200 can also include one or more network interfaces 1208 for communicating via one or more networks, such as Ethernet adapters, wireless transceivers, or serial network components, for communicating over wired or wireless media using protocols. Computer device 1200 can also include one or more storage devices 1210 of varying physical dimensions and storage capacities, such as flash drives, hard drives, random access memory, etc., for storing data, such as images, files, and program instructions for execution by one or more processors 1202.

Additionally, computer device 1200 can include one or more software programs 1212 that enable the functionality described above. One or more software programs 1212 can include instructions that cause one or more processors 1202 to perform the processes, functions, and operations described herein, for example, with respect to the processes of FIG. 1, FIG. 2A and FIG. 2B. Copies of one or more software programs 1212 can be stored in one or more memory devices 1204 and/or on in one or more storage devices 1210. Likewise, the data utilized by one or more software programs 1212 can be stored in one or more memory devices 1204 and/or on in one or more storage devices 1210.

In implementations, computer device 1200 can communicate with other devices via network 1214. The other devices can be any types of devices as described above. Network 1214 can be any type of network, such as a local area network, a wide-area network, a virtual private network, the Internet, an intranet, an extranet, a public switched telephone network, an infrared network, a wireless network, and any combination thereof. Network 1214 can support communications using any of a variety of commercially-available protocols, such as TCP/IP, UDP, OSI, FTP, UPnP, NFS, CIFS, AppleTalk, and the like. Network 1214 can be, for example, a local area network, a wide-area network, a virtual private network, the Internet, an intranet, an extranet, a public switched telephone network, an infrared network, a wireless network, and any combination thereof.

Computer device 1200 can include a variety of data stores and other memory and storage media as discussed above. These can reside in a variety of locations, such as on a storage medium local to (and/or resident in) one or more of the computers or remote from any or all of the computers across the network. In some implementations, information can reside in a storage-area network (“SAN”) familiar to those skilled in the art. Similarly, any necessary files for performing the functions attributed to the computers, servers, or other network devices may be stored locally and/or remotely, as appropriate.

In implementations, the components of computer device 1200 as described above need not be enclosed within a single enclosure or even located in close proximity to one another. Those skilled in the art will appreciate that the above-described componentry are examples only, as computer device 1200 can include any type of hardware componentry, including any necessary accompanying firmware or software, for performing the disclosed implementations. Computer device 1200 can also be implemented in part or in whole by electronic circuit components or processors, such as application-specific integrated circuits (ASICs) or field-programmable gate arrays (FPGAs). Some of these microprocessors may include dedicated neural network hardware capability specifically designed to implement artificial neural networks and RNNs described here.

If implemented in software, the functions can be stored on or transmitted over a computer-readable medium as one or more instructions or code. Computer-readable media includes both tangible, non-transitory computer storage media and communication media including any medium that facilitates transfer of a computer program from one place to another. A storage media can be any available tangible, non-transitory media that can be accessed by a computer. By way of example, and not limitation, such tangible, non-transitory computer-readable media can comprise RAM, ROM, flash memory, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, includes CD, laser disc, optical disc, DVD, floppy disk and Blu-ray disc where disks usually reproduce data magnetically, while discs reproduce data optically with lasers. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Combinations of the above should also be included within the scope of computer-readable media.

The foregoing description is illustrative, and variations in configuration and implementation can occur to persons skilled in the art. For instance, the various illustrative logics, logical blocks, modules, and circuits described in connection with examples of the present disclosure disclosed herein can be implemented or performed with a general purpose processor, a digital signal processor (DSP), an application specific integrated circuit (ASIC), a field programmable gate array (FPGA), cryptographic co-processor, or other programmable logic device, discrete gate or transistor logic, discrete hardware components, or any combination thereof designed to perform the functions described herein. A general-purpose processor can be a microprocessor, but, in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

In one or more examples, the functions described can be implemented in hardware, software, firmware, or any combination thereof. For a software implementation, the techniques described herein can be implemented with modules (e.g., procedures, functions, subprograms, programs, routines, subroutines, modules, software packages, classes, and so on) that perform the functions described herein. A module can be coupled to another module or a hardware circuit by passing and/or receiving information, data, arguments, parameters, or memory contents. Information, arguments, parameters, data, or the like can be passed, forwarded, or transmitted using any suitable means including memory sharing, message passing, token passing, network transmission, and the like. The software codes can be stored in memory units and executed by processors. The memory unit can be implemented within the processor or external to the processor, in which case it can be communicatively coupled to the processor via various means as is known in the art.

While the preferred embodiments have been shown and described, it will be understood that there is no intent to limit the invention by such disclosure, but rather, is intended to cover all modifications and alternate constructions falling within the spirit and scope of the invention.

1. A method for cochlear implant processing, the method comprising:
receiving, at a receiver of a cochlear implant, an input natural sound pattern;
processing, by a front-end processing algorithm, the input natural sound pattern to produce a target population firing pattern for a cochlea; and
transforming, by a back-end processing algorithm, the target population firing pattern to a simulation pattern that induces a response with naturalistic timing.

2. The method of claim 1, wherein the front-end processing algorithm comprises a trained neural network.

3. The method of claim 2, wherein the trained neural network is a trained recurrent neural network.

4. The method of claim 3, wherein the trained recurrent neural network is trained to learn a sound wave-to-spiking relationship of a phenomenological model of the cochlea.

5. The method of claim 4, wherein the phenomenological model of the cochlea accounts for outer hair cell and inner hair cell contributions to firing, filtering effects, and non-linearities related to synaptic and axonal activation.

6. The method of claim 3, wherein the trained recurrent neural network is trained on a synthetic waveform data set and a speech command dataset.

7. The method of claim 3, wherein the trained recurrent neural network transforms sound pressure level (SPL) into spiking and firing rate over time for an auditory nerve fiber with low, medium, or high spontaneous firing.

8. A system for cochlear implant processing, the system comprising:
a cochlear implant comprising a receiver that receives an input natural sound pattern;
a front-end processing algorithm that processes the input natural sound pattern to produce a target population firing pattern for a cochlea; and
a back-end processing algorithm that transforms the target population firing pattern to a simulation pattern that induces a response with naturalistic timing.

9. The system of claim 8, wherein the front-end processing algorithm comprises a trained neural network.

10. The system of claim 9, wherein the trained neural network is a trained recurrent neural network.

11. The system of claim 10, wherein the trained recurrent neural network is trained to learn a sound wave-to-spiking relationship of a phenomenological model of the cochlea.

12. The system of claim 11, wherein the phenomenological model of the cochlea accounts for outer hair cell and inner hair cell contributions to firing, filtering effects, and non-linearities related to synaptic and axonal activation.

13. The system of claim 10, wherein the trained recurrent neural network is trained on a synthetic waveform data set and a speech command dataset.

14. The system of claim 10, wherein the trained recurrent neural network transforms sound pressure level (SPL) into spiking and firing rate over time for an auditory nerve fiber with low, medium, or high spontaneous firing.

15. A method for neural implant processing, the method comprising:
receiving, at a receiver of a neural implant, an input activation pattern;
processing, by a front-end processing algorithm, the input activation pattern to produce a target population firing pattern for one or more neurons; and
transforming, by a back-end processing algorithm, the target population firing pattern to a simulation pattern that induces a response with naturalistic timing.

16. The method of claim 15, wherein the neural implant comprises a cochlear implant, a vestibular implant, a retinal vision prostheses, a deep brain stimulator, or a spinal cord stimulator.

17. The method of claim 15, wherein the front-end processing algorithm comprises a trained neural network.

18. The method of claim 17, wherein the trained neural network is a trained recurrent neural network.

19. The method of claim 17, wherein the trained neural network is trained using clinical data, a phenomenological model, or both.

20. The method of claim 17, wherein the trained neural network comprises one or more convolution layers for retinal prosthesis analysis.

21.-26. (canceled)
